(19) United States
(12) Patent Application Publication
(10) Pub. No.: US 2026/0000738 A1
(43) Pub. Date: Jan. 1, 2026

(54) STABILIZED FORMULA OF CROTOXIN

(71) Applicant: Celtic Biotech Ltd., Dublin (IE)

(72) Inventor: Paul REID, Chuluota, FL (US)

(73) Assignee: Celtic Biotech Ltd.q, Dublin (IE)

(21) Appl. No.: 18/855,454

(22) PCT Filed: May 19, 2024
(86) PCT No.: PCT/US2024/030129

§ 371 Date: Dec. 31, 2024

Related U.S. Application Data
(60) Provisional application No. 63/503,190, filed on May 19, 2023.

(51) Int. Cl. A61K 38/46; A61K 47/02; A61K 47/18

(52) U.S. Cl. CPC A61K 38/465; A61K 47/02; A61K 47/186; C12Y 301/01004

(57) ABSTRACT

A highly stable acidifed formula of Crotoxin comprising saline and benzalkonium chloride, and optionally Thiamine, is disclosed that facilitates the therapeutic use of Crotoxin. Also disclosed is a method of preparing a highly stable formula of Crotoxin.

### FIELD OF THE INVENTION

The invention relates to a robust formulation of Crotoxin for clinical administration that allows for easy handling and reduced waste. Also disclosed is a method for formulating a solution of Crotoxin that provides the aforementioned attributes.

### BACKGROUND OF THE INVENTION

Crotoxin was first isolated in 1934 and has been extensively researched because it represents the main toxic component of Crotalus durissus venom. Its neurotoxicity is attributed to its high affinity for nicotinic acetylcholine receptors located on the diaphragm of the envenomed animal. The protein's A subunit (Crotapotin) suppresses the enzymatic activity of its B subunit (Crotactine) in addition to targeting the protein to its receptor. The two subunits are attached through covalent forces, which can be separated under specific conditions. The two subunits will spontaneously associate when recombined either in-vitro or in-vivo. It was discovered that Crotoxin was highly toxic to cancerous cells both in-vitro and in-vivo, leading to human clinical studies in subjects with cancer. Subsequently, it was found that Crotoxin exerted additional pharmacodynamic properties: having anti-inflammatory and antiviral activity. Storage and administration of protein-based oncology drugs is often difficult and can lead to significant losses of drug material though improper packaging and handling. Estimates report that these costs range from $1.8 to $3.0 billion annually. The application describes a formulation of Crotoxin that permits the simple storage and administration of the drug which can avoid unnecessary waste.

A known method for Crotoxin purification is to suspend crude venom in water and adjust the pH to 2.0 with dilute HCl. The preparation is then heated at 90° C. for 10 min and centrifuged. The supernatant is removed and the pH of the supernatant is adjusted to 4.7 (with diluted ammonia) to cause a precipitate. The pH-adjusted supernatant and ammonia solution is centrifuged and the precipitate retained, which contains mostly Crotoxin. There are several disadvantages to this method: 1. Not all Crotoxin is recovered from the crude venom, 2. The recovered Crotoxin is not high purity and 3. Crotoxin's activity is reduced by the heat process. The advantages are: 1. Precipitation of extraneous proteins, 2. Killing of viral or adventitious contaminants and 3. It is fast.

U.S. Pat. No. 5,164,196 and EP 0246861 disclose a stable composition of matter based on the cytotoxic activity of a Crotoxin B. Venom is dissolved in ammonium formate at pH 4.0 and the Crotoxin peak is isolated by size exclusion chromatography (one run on G75 and one on S200 resins). The eluant fractions corresponding to the main cut are combined (about 25 ml containing 23-25 mg protein), filtered (0.22 μm pore size), concentrated to a volume of 5-7 ml using ultrafiltration in a 50 ml Amicon cell fitted with a Filtron membrane Omega, mol. wt. limit 3K), filter-sterilized again (0.22 μm pore size) and lyophilized.

Lyophilized Crotoxin is dissolved in Phosphate buffer (35 mM KHPO4, pH7.0) and the solution loaded on a DEAE column. Elution of Crotoxin is achieved with NaCl gradient up to 1.0 M. The Crotoxin heterodimer elutes as a relatively broad peak from anion exchange resins due to the existence of isoforms. The resulting DEAE Crotoxin solution is diafiltered into 50 mM ammonium formate, pH 4.5, which is volatile and lyophilized. Typically, 250 mg of venom is purified in this way. The inventors concluded that ion-exchange chromatography was deemed unsuitable as a routine first step for purification because it is a time-consuming process and it is not a large-scale process. However, the process does yield a pure protein.

Other known methods use organic acids to assist in dissociating the protein into its constituent subunits to aide in separating the purification process. Organic acids such as citric, acetic and formic acid can facilitate this process, with the residual acidic buffers being removed through dialysis or lyophilization and suspended into a neutral pH 6.9 phosphate buffer.

Reverse phase chromatography may also be used for purification but this technique also causes the dissociation of the subunits in an organic solution that is not readily compatible with drugs destined for injection. Additional considerations for these processes are the maintenance of cleanliness of the production processes, avoiding microbial or endotoxin contamination that could compromise the final product.

The use of animal derived products also requires consideration of contamination with adventitious viral agents, usually viral or mycoplasma, that must be removed or inactivated during the production process. Because monoclonal antibodies are made using animal-derived cell lines, current standard practice for monoclonal antibody production includes reducing the pH to 4.0 for a period of several hours followed by filtration through membranes designed to retain viruses. The World Health Organization's 2004 “Annex 4 Guidelines on viral inactivation and removal procedures intended to assure the viral safety of human blood plasma products” recognizes that most proteins are damaged by exposure to the acidic conditions needed to kill viruses. For example, few viruses are killed at pH 5.0-5.5, a condition known to inactivate factor VIII. Immune globulin solutions are an exception. Various studies have shown that low pH, such as in the pH 4 treatment used in the preparation of immunoglobulins, inactivates several enveloped viruses. On the basis of these and other results, the WHO guideline suggests one manufacturer may incubate its immunoglobulin preparation at pH 4.0 for at least 6 hours at 37° C. whereas another follows solvent/detergent treatment by incubating in the container at pH 4.25 for a minimum of 21 days at 20° C. In either process, the pH is never less than 4.0 and no longer than 30 days.

Previously, antibodies are reported to be quite robust and should retain functional activity if kept refrigerated at 2-8° C. for up to 12 months. For longer term storage, it is recommended to store the antibodies frozen in smaller aliquots and to avoid repeated freezing and thawing cycles, which adversely affects the functional activity of the antibodies. Generally, antibodies can be left at room temperature for up to a week without loss of activity. Hence, antibodies may be shipped at ambient temperature. However, longer storage at room temperature is not recommended, as there is no guarantee of the performance of the antibody under such circumstances. Current monoclonal antibody formulations often use sucrose to stabilize the concentrated protein for refrigerated or frozen storage with shelf lives up to 3 years. However, storage recommendations for the monoclonal antibody, pembrolizumab, by the manufacturer advise storage at room temperature for no more than 6 hours from the time of dilution. This includes room temperature storage of the diluted solution, and the duration of infusion. Storage is advised under refrigeration at 2° C. to 8° C. (36° F. to 46° F.) for no more than 96 hours from the time of dilution. Similar guidance is provided by the manufacturers of the monoclonal antibody, Atezolizumab, for which the chemical and physical in-use stability of diluted product has been demonstrated for up to 24 hours at ≤30° C. and for up to 30 days at 2° C. to 8° C. from the time of dilution.

Previously, oral administration of Crotoxin was reported to be ineffective, presumably because the protein could not survive the acidic conditions of the stomach, a concept that is standard dogma. HCl is the primary acid in the stomach.

### BRIEF SUMMARY OF THE INVENTION

A formulation of Crotoxin exhibiting stability comprising a saline solution of about 0.9% and a pH of about 1.5 to about 4.5 is disclosed, wherein the formulation is acidified using HCl. In one embodiment, the stable formulation of Crotoxin exhibits stability under refrigerated conditions for up to 106 months at a temperature of between about 1-10° C. comprising a pH of about 3.5 to about 4.5, wherein the concentration of Crotoxin as measured at A280 nm absorbance is about 350 to about 403 μg/mL. In one embodiment, the stable formulation of Crotoxin further comprises about 0.009% benzalkonium chloride. In one embodiment, the stable formulation of Crotoxin further comprises about 0.01 to 0.1% Thiamine.

### DETAILED DESCRIPTION OF THE INVENTION

A formulation of Crotoxin is disclosed that is stable under normal refrigerated conditions for drug storage over long periods of time and also exhibits exceptional room temperature stability. A robust formulation of Crotoxin for clinical administration that allows for easy handling and reduced waste should allow the protein stay in solution, retain its biological activity without dissociation and be compatible with a preservative to allow a multi-use vial format.

According to one embodiment of the invention, Crotoxin was found to be stable in a saline solution for long periods even at an acid pH of less than 2.0, when the pH was lowered using Hydrochloric acid (HCl). According to one embodiment of the invention, it was found that Crotoxin could remain active when stored for long periods at a pH of 1.5 in a simple solution of 0.9% saline using HCl to lower the pH. Additionally, when using ultraviolet absorption to help estimate the protein concentration in solution, it was observed that a reduction in the absorbance levels of the protein at 280 nm occurred when Crotoxin is in low pH solutions. It was thought that the absorbance changes resulting from the low pH was likely a consequence of structural changes that compact the size of the protein thereby concealing amino acids contributing to the absorbance levels. These absorbance changes were most obvious in saline solutions with a pH lower than 4.0. Being aware of the pH-induced structural effects and in spite of reservations as to it success, it was thought that this structural change may serve to protect amino acids within the protein from oxidation thereby protecting the integrity of the critically important phospholipase enzyme. As a result, while storage at acidic conditions is detrimental to the vast majority of proteins, it was believed that a solution formulation with a pH of 2.5-4.0 would have long-term stability allowing the facile storage and use of Crotoxin.

Crotoxin is not soluble in water unless it is acidic (<3.0). It is reported that the subunits of Crotoxin dissociate at a pH of less than 2.5 but this depends on the solution in which it is stored. At higher pH levels, generally above 4.0, the presence of salts is required at a minimum of 35 mM to have Crotoxin remain in solution. Even then, Crotoxin can precipitate out of solution (at or around a pH 4 or above) and the pH must be adjusted to below 2.5 or above 9.5 for it to dissolve again. Changing pH is a delicate operation unless high salt (>0.1 M) is present. Certain buffers such as citrate solutions can cause dissociation of the Crotoxin subunits at a pH of less than 4.0, which makes the administration of the product as a drug undesirable because any free crotactine can lyse cells at the site of injection or erythrocytes if injected intravenously.

Starting with a range of formulations using saline (pH 5.0) and phosphate buffered saline (pH 6.9-7.2), an accelerated study of stability of Crotoxin was completed at 49° C. This temperature is higher than normal accelerated temperatures used in standard stability testing, which is usually 42° C., because Crotoxin has an inherent stability at elevated temperature and can survive boiling for 10 min above 95° C. It was determined that Crotoxin was more stable at the lower pH of 5.0 as protein precipitation was observed and A280 value declined. Two preservatives were assessed under accelerated conditions; methyl paraben and benzalkonium chloride. Prior testing had eliminated the use of propylene glycol as a preservative as it seemed to interfere with the potency of Crotoxin. Solutions with methyl paraben accelerated protein precipitation and were rejected in favor of benzalkonium chloride.

Similar observations were made when stored at room temperature with deterioration manifesting with increased protein precipitation. The potency of the drug declined in step with visual changes to the solution, although the lower pH solution retained its potency specification for over 24 months versus 12-18 months when the protein was stored at a more neutral pH.

When stored refrigerated (1-10° C.) the potency of Crotoxin has been maintained for over 10 years formulated as ready-to-use in 0.9% saline at a pH of 3.0-4.0, using HCl for acidification, and benzalkonium 0.009chloride (approximating to 0.009%) as the system preservative. Over this time, periodic exposure to room temperature has had no detectable impact on the activity of the drug. During formulation, the addition of benzalkonium chloride causes the solution to go cloudy but the solution clarifies if allowed to stand at room temperature for 24 hrs. It is also thought that the cationic charge of the preservative interacts with the protein and contributes to the stability of the protein solution by preventing aggregation of protein by blocking protein-protein interaction through charges associated with the constituent amino acids.

The build-up of protein charges can result from oxidative reactions with hydroxyl ions occurring in water. It has been found that an excellent antioxidant neutralizer of these radicals is the vitamin, Thiamine, which can be incorporated into this formulation in the range of 0.01 to 0.1%, thereby enhancing the formula's stability even further. When exposed to high temperatures, thiamine was found to protect peptides from oxidation particularly the readily reduced sulfur amino acids, methionine and cysteine.

In one embodiment, the formula comprises about 0.9 wt. % saline. In one embodiment, the formula comprises about 0.9 vol. % saline. In one embodiment, the formulation comprises about 0.009 wt. % benzalkonium chloride. In one embodiment, the formulation comprises about 0.009 vol. % benzalkonium chloride. In one embodiment, the stable formulation of Crotoxin further comprises about 0.01 to 0.1 wt. % Thiamine. In one embodiment, the stable formulation of Crotoxin further comprises about 0.01 to 0.1 vol. % Thiamine.

The resulting formula has several advantages. The low pH assists in preventing the protein solution from becoming grossly contaminated with microbes, in addition to deactivating any adventitious agents. The preservative, as a detergent, is also known to inactivate enveloped viruses further protecting the patient from such agents, while protecting the solution from contamination with repeated needle entries. Lastly, the simple storage conditions provide confidence in its handling and shipping and should contribute to minimizing drug wastage.

Table 1 shows testing results conducted over a period of 106 months of Crotoxin with benzalkonium chloride in a 0.9% saline solution stored at refrigerated conditions of 2-8° C.

TABLE 1

| Time | | Clarity | | | Concentration | Potency | Preservative |
|---|---|---|---|---|---|---|---|
| Point | | (No visible | MW/Integrity | pH | (A280 nm) | (Mouse, | (HPLC, |
| (month) | Appearance | particulates) | (SEC) | (Litmus) | μg/mL | min) | BZK) |
| 0 | colorless | Clear, no | 24 kD/Pass | 4.5 | 403 | 60 | 0.0088% |
| | | PPT | | | | | |
| 3 | colorless | Clear, no | 24 kD/Pass | 4.5 | 387 | 123.7 | 0.0089% |
| | | PPT | | | | | |
| 6 | colorless | Clear, no | 24 kD/Pass | 4.5 | 401 | 91 | 0.0092% |
| | | PPT | | | | | |
| 9 | colorless | Clear, no | 24 kD/Pass | 4.5 | 408 | 125.7 | 0.0092% |
| | | PPT | | | | | |
| 12 | colorless | Clear, no | 24 kD/Pass | 4.5 | 388 | 92.5 | 0.0095% |
| | | PPT | | | | | |
| 18 | colorless | Clear, no | 24 kD/Pass | 4.5 | 378 | 148.7 | 0.0098% |
| | | PPT | | | | | |
| 27 | colorless | Clear, no | 24 kD/Pass | 4.5 | 410 | 89 | 0.009% |
| | | PPT | | | | | |
| 33 | colorless | Clear, no | 24 kD/Pass | 4.5 | ND | 84 | ND |
| | | PPT | | | | | |
| 46 | colorless | Clear, no | 24 kD/Pass | 4.0 | ND | 110 | ND |
| | | PPT | | | | | |
| 62 | colorless | Clear, no | 24 kD/Pass | 4.0 | 400 | 77 | ND |
| | | PPT | | | | | |
| 70 | colorless | Clear, no | 24 kD/Pass | 4.0 | 400 | 78.5 | ND |
| | | PPT | | | | | |
| 82 | colorless | Clear, no | 24 kD/Pass | 3.36 | 374 | 61.3 | ND |
| | | PPT | | | | | |
| 94 | colorless | Clear, no | 24 kD/Pass | 3.35 | 350 | 83 | ND |
| | | PPT | | | | | |
| 106 | Colorless | Clear, no | 24 kD/Pass | 3.5 | 375 | 74.6 | ND |
| | | PPT | | | | | |

ND: Not Done

1-5. (canceled)

6. A formulation of Crotoxin, exhibiting stability comprising about 0.9 wt. % saline solution, about 0.009 wt. % benzalkonium chloride and a pH of about 1.5 to about 4.5, wherein the formulation is acidified using HCl, wherein the concentration as measured at A280nm absorbance comprises about 350 to about 375 μg/mL after 106 months storage at about 2-10° C.

7-9. (canceled)

10. A formulation of Crotoxin, exhibiting stability comprising about 0.9 wt. % saline, about 0.009 vol. % benzalkonium chloride and a pH of about 1.5 to about 4.5, wherein the formulation is acidified using HCl, wherein the concentration as measured at A280nm absorbance comprises about 350 to about 375 μg/mL after 106 months storage at about 2-10° C.

11-14. (canceled)

15. A formulation of Crotoxin, exhibiting stability comprising about 0.9 wt. % saline solution, about 0.009 wt. % benzalkonium chloride, about 0.009 vol. % benzalkonium chloride and a pH of about 1.5 to about 4.5, wherein the formulation is acidified using HC1, wherein the formulation exhibits stability under refrigerated conditions for up to 106 months at a temperature of between about 1-10° C. comprising a pH of about 3.5 to about 4.5, wherein the concentration as measured at A280nm absorbance comprises about 350 to about 375 μg/mL after 106 months storage at about 2-10° C.

16-18. (canceled)

19. A formulation of Crotoxin, exhibiting stability comprising about 0.9 wt. % saline solution, about 0.009 vol. % benzalkonium chloride and a pH of about 1.5 to about 4.5, wherein the formulation is acidified using HCl, wherein the formulation exhibits stability under refrigerated conditions for up to 106 months at a temperature of between about 1-10° C. comprising a pH of about 3.5 to about 4.5, wherein the concentration as measured at A280nm absorbance comprises about 350 to about 375 μg/mL after 106 months storage at about 2-10° C.

20-21. (canceled)
